(19) United States
(12) Patent Application Publication
(10) Pub. No.: US 2003/0138861 A1
(43) Pub. Date: Jul. 24, 2003

(54) Method for diagnosis of disease state of the bilio-pancreatic system

(76) Inventor: Jens Brockmann, Munster (DE)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/181,956

(22) PCT Filed: Feb. 5, 2001
(86) PCT No.: PCT/EP01/01195

(30) Foreign Application Priority Data
Feb. 4, 2000 (DE) 100 04 919.2

(51) Int. Cl. G01N 33/574

(52) U.S. Cl. 435/7.23

(57) ABSTRACT

The invention relates to trypsin activating peptide as a diagnostic agent for malignant bilio-pancreatic disease states.

The invention pertains to a method for diagnosing diseases of the bilio-pancreatic system, especially of carcinomas. It claims the priority of the German patent application 100 04 919, which is referred to in respect of disclosure.

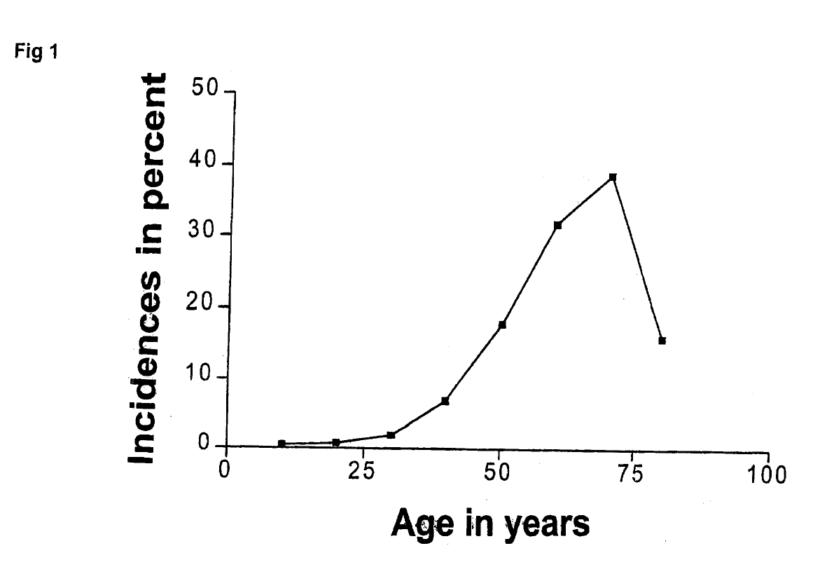

The organs of the so-called bilio-pancreatic system, namely the pancreas, the gall-bladder with the bile ducts as well as the papilia Vateri as a joint opening of the ductus coledochus coming from the liver and gall-bladder and the ductus pancreaticus coming from the pancreas into the duodenum, originate from a bud of the duodenal primordium during embryogenesis. In these organs, malignant tumours can develop, which—with increasing incidence—constitute a frequent cause for death of humans. Meanwhile each fourth case of death is due to malignant neoformation of the pancreatic cancer. Although carcinoma of the pancreas have also been described in neonates, this cancer is typical for elder patients (7,8). FIG. 1 depicts the typical age distribution for indices of pancreatic cancer. Bile-duct cancer is supposed to be still the fifth most frequent cause for deaths due to cancerous diseases.

With the exception of the papillary carcinoma, i.e. the tumour of the papilla Vateri, the carcinomas of the bilio-pancreatic system commonly cause rather unspecific symptoms such as a general drop in fitness, weight loss or painless symptoms as e.g. a painless jaundice (icterus). Thus, these carcinoma are detected and diagnosed rather late. In contrast, the papillary carcinoma can be diagnosed earlier. This difference might be due to the exposed anatomic position of the papilla Vateri, causing early striking symptoms with tumour formation and consequently allowing earlier diagnosis and better prognosis. In case of other carcinomas of the bilio-pancreatic system and their late diagnosis only few patients can be treated with curative operations. For example, only 5-20% of patients with pancreatic cancer and only 30% of patients with a bile-duct cancer can be operated (9). When diagnosed only 20% of the gall-bladder carcinoma are still limited to this organ, thus only 20% of all patients with gall-bladder carcinoma can be curatively operated.

Even after being curatively operated patients face only low chances for survival. Thus, the survival ratio of patients with a gall-bladder carcinoma in the first 5 years after operation is less than 5%. With bile-duct cancer, the 5-years-survival-rate varies between 0 and 58%.

Since the operative removal (tumour resection) currently constitutes the only curative therapy, an early diagnosis (the so-called screening) is essentially important. However, as already mentioned, due to more unspecific and painless symptoms an early diagnosis is very difficult. The screening of pancreatic cancer is even impaired due to the special problem of differentiating between a pancreatic carcinoma and a chronic pancreatitis, since both pancreatic diseases cause similar clinical symptoms. Often, even after an operation and a pathohistological inspection, a distinction is not possible. Instead, only the further clinical progress (death, survival) allows for a clear evaluation.

The evaluation of dignity, i.e. the differentiation between an inflammatory benignant and a malignant pancreatic disease, is furthermore important due to the supposed—but not yet finally proofed—causal connection between the chronic pancreatitis and the pathogenesis of the carcinoma. Thus, studies confirmed, that the hereditary pancreatitis correlates to a standardised risk of 53 for cancerous transformation.

Also the pathogenesis of the other cancers of the bilio-pancreatic system is probably enhanced by chronic inflammations. Thus, the bile-duct or gall-bladder cancer often correlates with chronic inflammatory diseases of the colon (colitis ulcerosa). Furthermore, over 80% of the patients with malignant tumours suffer from gall-stones, which can cause chronic inflammations as such.

For early diagnosis it is known to perform blood analyses for tumour markers such as CA 19-9, CA 242, CEA, CA 72-5, CA 125 or CA 50. These tests can reach a sensitivity of up to 80% and a specificity of up to 90%. However, the sensitivity of the previously used tumour marker analyses is negatively influenced by other diseases correlating with jaundice (3-5). Furthermore, in case of lacking of a special blood group antigen —namely the Lewis-blood group antigen a —CA 19-9 is not detectable (6). CA 19-9 is far the most common tumour marker.

Other approaches to improve early diagnosis are based on employing thin layer-computer tomography or endosonography, i.e. the ultrasound in the interior of the body. However, they are more suitable for confirming a diagnosis and exploring the operative situation, than constituting a suitable screening method as such. Even multicenter studies with already identified patients were not useful in order to develop recommendations for screening methods (2).

Thus, it is an object of the invention to provide a method for early stage diagnosing carcinomas of the bilio-pancreatic system.

This object is achieved by a method according to claim 1. Advantageous embodiments are subject of the dependent claims.

The invention is based on the idea of detecting malignant tumours in the bilio-pancreatic system by quantifying the trypsinogen activating peptide (designated also as TAP) in body fluids. Thus, the method according to the invention is based on using an increased TAP-concentration as a marker for bilio-pancreas carcinomas. Consequently it prerequisites a correlation between an increased TAP-concentration and the occurrence of malignant tumours.

Among experts the correlation between the occurrence of bilio-pancreas carcinomas and an increased TAP-concentration was not only un- recognised. It sometimes even was negated, since this correlation contravenes commonly accepted theories—which are accepted nearly for a century—about the pathenogensis of the pancreas carcinoma. This is due to reasons outlined below:

For the later enzymatic digestion of proteins and polypeptides within the small bowel, within the pancreas trypsinogen as an inactive precursor (zymogen) of the protein-cleaving enzyme trypsin is produced. Therewith trypsinogen is secreted into the duodenum via the ductus pancreaticus at the joint opening with the ductus coledochus coming from the gall-bladder and the liver. Here, trypsinogen is activated to trypsin by a hydrolytically active enterokinase (enteropeptidase) produced in the intestinal mucosa.

By hydrolysis of trypsinogen besides trypsin, tetra-L-aspartil-L-lysine is released. Due to its trypsinogen activating properties, this peptide is called “trypsinogen activating peptide” (TAP). It is a pentapeptide with a molecular weight of 0,606 kDA, which exhibits the following function for the formation of a severe acute necrotic pancreatitis:

In case of passing of a gall-bladder stone, a small gall-bladder stone travels from the gall-bladder into the bile-ducts and sticks at the papilla Vateri, i.e. the joint opening for the bile and pancreas secretion. Consequently, the duct to the duodenum is blocked, which—according to the previous models—induces a reflux of the accumulated bile secretion into the pancreas (14). Within the pancreas, the inactive enzyme precursors—thus also the trypsinogen—mainly remain in their inactive state. However, the active amylases and lipases also being present within the bile secretion induce an oedematic pancreatitis. In case also the inactive enzyme precursors are activated, i.e. in case of a zymogen-activation, severe acute necrotic pancreatitis is induced.

Based on this model, a method for differentiating between the oedematic and the necrotic pancreatitis was developed (U.S. Pat. No. 4,948,723). It is based on the immunological detection of TAP in body fluids, which is not present in case of oedematic pancreatitis, but detectable in higher amounts in case of necrotic pancreatitis.

Thus, this model explains the acute pancreatitis by a reflux of bile secretion into the pancreas, which sometimes coincides with a zymogen activation. Consequently, a correlation between the zymogen activation and the pathogenesis of gall-bladder and bile duct inflammation is excluded, since—according to the model—the zymogenes in the pancreas secretion is not in contact with these organs. The same applies for the papillary carcinoma.

Furthermore, according to this common theory, the genesis of a pancreatic carcinoma does not coincide with an activation of the pancreatic zymogenes. Thus, also the hydrolysis of trypsinogen to active trypsin and TAP does not correlate with pancreatic carcinoma.

With this model it could not reasonably be expected, that TAP would suit as an indicator for the occurrence of bilio-pancreas carcinomas. Instead, the teaching according to the invention first requires a rejection of the theory, which was accepted for a long time.

The teaching according to the invention is based on the assumption, that in case of an occlusion of the papilla Vateri a reflux of pancreatic secretion into the bile-ducts and not—as previously thought—an exclusive reflux of bile secretion into the pancreas occurs. Due to the entero-hepatic circulation, the bile secretion contains enterokinases, which can activate trypsinogen and thereby release TAP. The activated pancreatic enzymes can thus induce an inflammation in the bile-ducts, leading to local damages of the healthy tissue. Consequently, the inflamed cells can transform to malignant cells, leading to a possible occurrence of malignant tumours in the bile-ducts, the gall-bladder and in the region of the papilla Vateri.

This new model can also explain the pathogenesis of pancreatic cancer. It is based on the phenomenon, that the pressure in the bile-duct system normally is higher than in the excretory ducts of the pancreas. This is due to the stronger muscular system of the bile ducts. In case of constrictions or occlusions in the region of the papilla Vateri, for a short time in the pancreatic duct system a higher pressure can arise, since this organ—with a daily secretion of 3000 ml in comparison to 800 ml of bile secretion within the liver—secretes about 4-times more than the liver. Thus, trypsinogen can enter the bile-ducts, being activated there and—in the active form—flows back into the pancreas. There it can induce a proteolytic damage, possibly followed by a malignant cellular transformation.

Besides the diagnosis of inflammations of the gall-bladder, the bile-ducts and the papilla Vateri, the method according to the invention allows for a highly specific and sensitive diagnosis of bilio-pancreas carcinomas. They can be clearly distinguished from other bilio-pancreatic diseases, e.g. inflammations. Therefore, the teaching according to the invention provides for the possibility of early carcinoma diagnosis and respective early operative treatment.

Using the method according to the invention one can also favourably distinguish bilio-pancreas carcinomas from other gastrointestinal carcinomas.

The quantification of TAP is preferably performed by an immunological detection in a quantitative solid-phase-immunoassay. Further biochemical or physical quantitative detection methods are suitable as well.

The patient probes to be tested for their TAP-contents can be taken from various body fluids. Suitable are e.g. probes derived from blood, bile secretion or urine. The latter is due to the low molecular weight of TAP. It can, especially in case of a stasis in the bile ducts, enter the bloodstream. From the blood it can be renally filtered and is therefore detectable in the urine.

### EXAMPLE:

### 1. Patients

The bile secretion of 44 patients being consecutively cholecystectomised were analysed from July 1999 to November 1999. These patients showed the following diseases:

pancreas carcinoma; n=12 (30%)

carcinoma of the bile duct; n=1 (2%)

papillary carcinoma; n=2 (5%)

chronic cholecystolithiasis; n=14 (32%)

acute cholecystolithiasis; n=2 (5%)

chronic pancreatitis; n=6 (13%)

other tumour diseases coincided with cholecystolithiasis; n =6 (13%)

A total number of 16 patients with bilio-pancreatic carcinoma were examined.

The patients enlisted under 7. also suffered from a malignant disease of the intestinal tract, but the tumour was not localised in the bilio-pancreatic system.

### 2. Sampling

For TAP determination, bile secretion was perioperatively isolated from removed (ectomised) gall-bladders. A 18 G aspiration needle was used. Within the first two hours after aspiration the samples were centrifuged at 3000 g for 10 min in order to remove living cells like leucocytes or epithelian cells from the gall-bladder. The sample aliquots were stored at −80° C. before analysis. The gall-bladder samples were previously examined macroscopically and microscopically for pathogenic characteristics. For the subsequent examinations, only tumour free gall-bladders were used.

The results were correlated with the intra-operative findings and the histopathological results.

### 3. Quantification of TAP

The studies were performed using the commercially available test TAPKIT (Biotrin International Ltd., Dublin, Ireland). This test is known from the U.S. Pat No. 5,356,781, which is—as well as its parallel German laid-open specification 37 89 584—fully incorporated by reference.

The test TAPKIT for quantifying TAP is a quantitative solid-phase-enzyme-immunassay, based on the competitive binding of free and immobilised peptides to an anti-TAP-antibody. The selected antibody is specific for the carboxy-terminus of TAP and was synthesised by immunisation against TAP N-terminally coupled to thyreoglobulin in a rabbit.

The TAP contained within the probe is immobilised at a solid phase (plate) by a carrier molecule and incubated with rabbit-anti-peptide-antibodies with biotinlylated IgG and streptavidin-horseradish-peroxidase additive. Subsequently the plate is developed with tetramethyl-benzidine (TMD) and the TAP concentration is quantified. The resulting intensity of colour is indirectly proportional to the amount of TAP within the sample.

The range of measurement varied between 0,45-1000 nM. Probes were diluted if TAP-Values in the original samples exceeded this range.

### 4. Results

The TAP-concentrations within the bile secretion unambiguously indicate towards higher concentrations for carcinomas of the bilio-pancreatic system.

TABLE 1
Mathematical characterisation of the distinct disease groups

| | | | | | | | Other |
|---|---|---|---|---|---|---|---|
| | | | | | | | carcinomas |
| | | | | Chronic | acute | Chronic | with chron. |
| | Pancreatic | bile-duct | Papillary | cholecyst- | chole- | pancrea- | cholecysto- |
| Diagnosis | carcinoma | cancer | carcinoma | olithiasis | cystitis | titis | lithiasis |
| Patients | 13 | 1 | 2 | 14 | 2 | 6 | 6 |
| (n) | | | | | | | |
| Minimum | 83.69 | 2571 | 408.3 | 0.0320 | 60.14 | 0.0080 | 0.0440 |
| value | | | | | | | |
| 25% | 352.1 | | | 0.2280 | | 0.0920 | 0.0860 |
| percentile | | | | | | | |
| Median | 1410 | 2571 | 563.6 | 1.228 | 77.71 | 5.684 | 5.994 |
| 75% | 3157 | | | 9.866 | | 18.36 | 13.36 |
| percentile | | | | | | | |
| Maximum | 5133 | 2571 | 719 | 16.30 | 95.27 | 22.37 | 13.81 |
| Mean | 1885 | 2571 | 563.6 | 4.515 | 77.71 | 8.046 | 6.481 |
| Std. | 1797 | 0 | 155.3 | 1.52 | 17.56 | 3.825 | 2.675 |
| deviation | | | | | | | |
| Std. error | 498.5 | 0 | 155.3 | 1.52 | 17.56 | 3.825 | 2.675 |
| Lower. | 798.9 | | −1410 | 1.231 | −145.4 | −1.787 | −0.3957 |
| 95% | | | | | | | |
| confidence | | | | | | | |
| interval | | | | | | | |
| Upper | 2971 | | 2537 | 7.8 | 300.9 | 17.88 | 13.36 |
| 95% | | | | | | | |
| confidence | | | | | | | |
| interval | | | | | | | |

Comparing the benignant and malignant diseases of the bilio-pancreatic system let to the following results.

TABLE 2
Comparison of the TAP-concentration in the bile secretion of patients with
benignant and malignant bilio-pancreatic diseases.

| | Malignant diseases of the bilio- | Benignant diseases of the bilio- |
|---|---|---|
| | pancreatic system | pancreatic system |
| Diagnosis | (groups 1-3) | (groups 4-7) |
| Patients (n) | 16 | 28 |
| Minimal | 83.69 | 0.0008 |
| 25% Percentile | 427.0 | 0.272 |
| Median | 1328 | 2.018 |
| 75% Percentile | 2190 | 13.19 |
| Maximum | 5133 | 95.27 |
| Mean | 1763 | 10.92 |
| Std. Deviation | 1684 | 20.51 |
| Std. Error | 421.0 | 3.875 |
| Lower 95% confidence interval | 865.3 | 2.969 |
| Upper 95% confidence interval | 2660 | 18.87 |

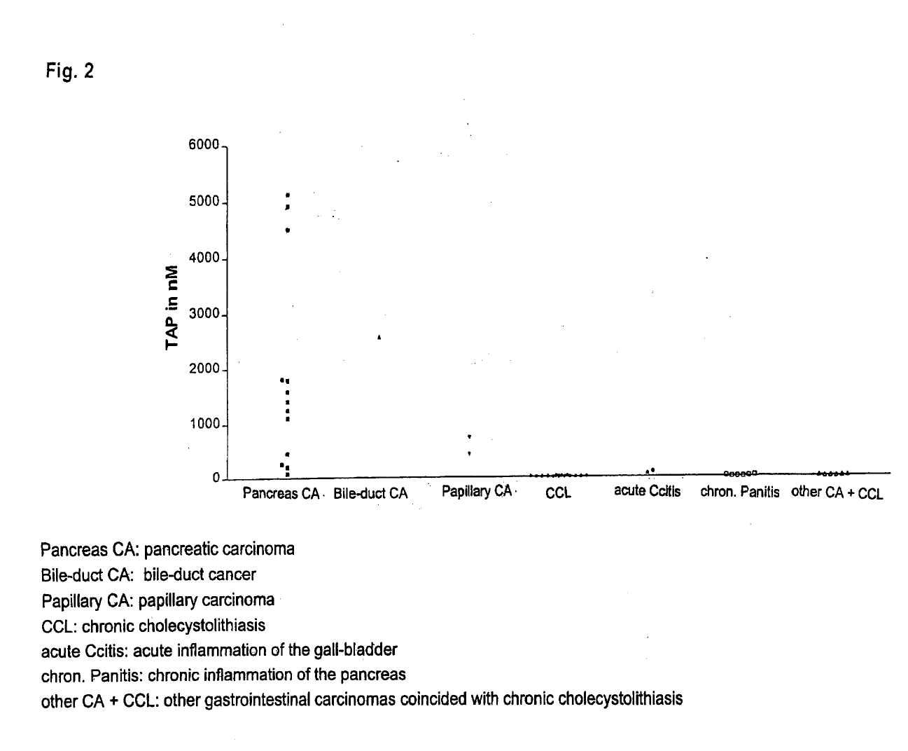

These results are shown in the FIGS. 2 to 5:

FIG. 2: Linear distribution of the TAP-concentration in bile secretion

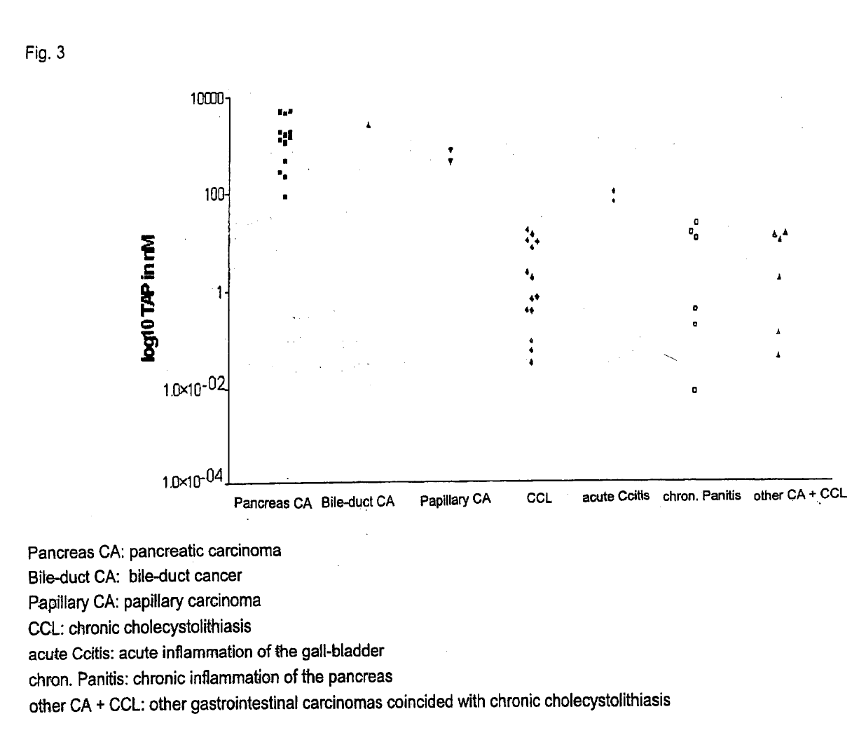

FIG. 3: Logarithmic (log10) distribution of the TAP-concentration in bile secretion

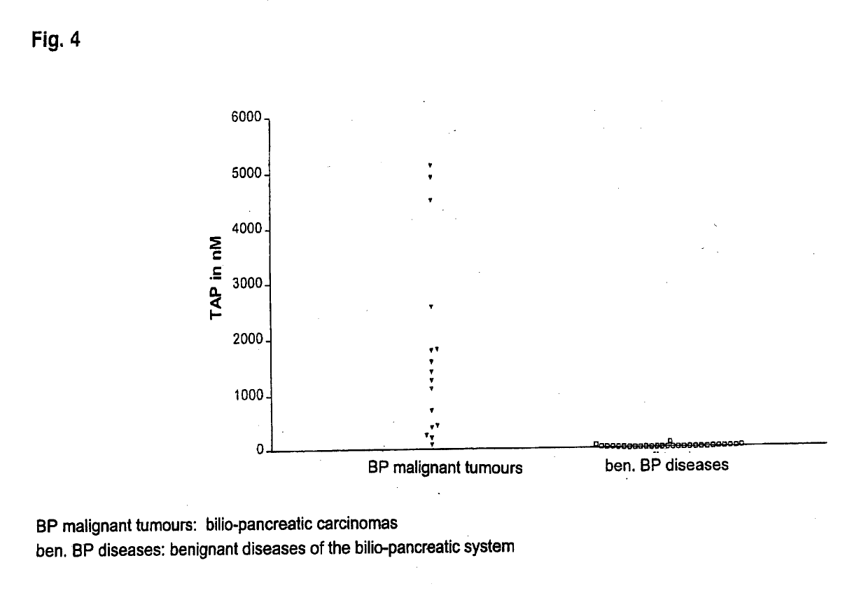

FIG. 4: Distribution of the TAP-concentration in bile secretion in linear depiction

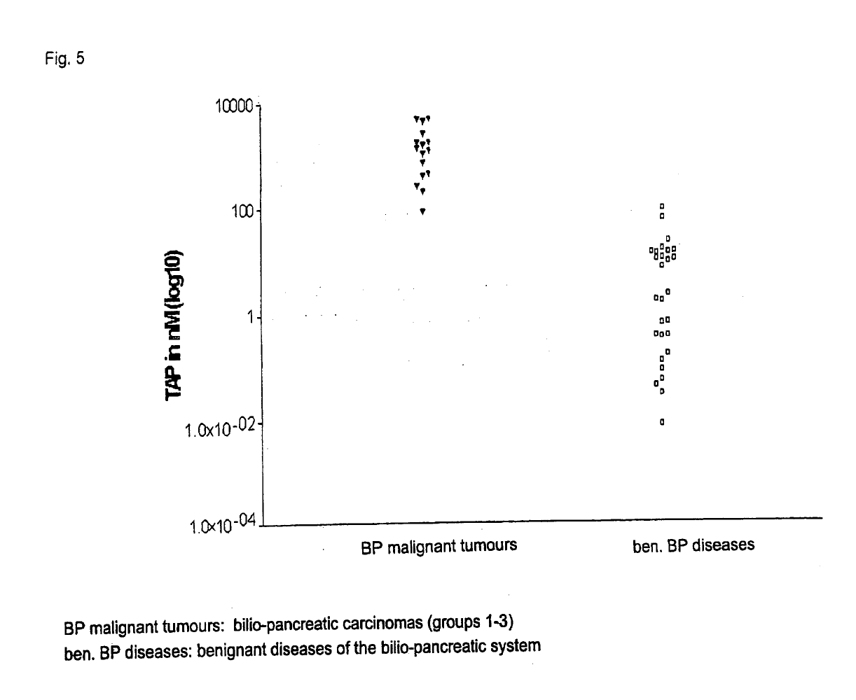

FIG. 5: Distribution of the TAP-concentration in bile secretion in logarithmic depiction

The statistical evaluation of these results—despite of the low patient number—can indicate a significant difference in the group comparison between malignant and benignant diseases of the bilio-pancreatic system. The Mann-Withney test for a non parametric analysis gave a P-value of >0,0001. Equally, in spite of a not displaying a Gauss distribution, the result of the unpaired t-tests was highly significant. The two-way ANOVA test yielded a highly significant discrimination between the two different groups.

Among the results, only one false positive value was found within the group of patients with acute gall-bladder inflammation (cholecystitis). This disease however is clinically so different, that a false positive result is of no practical relevance for the applicability of the method according to the invention. The false positive result further indicates, that—in comparison to a chronic inflammation like e.g. in gall-bladder stone diseases—an acute bile duct inflammation coincidences with an increased value for TAP in the gall-bladder duct system. However, especially this figure would verify the hypothesis of a reflux induced damage of the bile ducts.

In practice, the occurrence of a false positive value can be prevented by an increase of the cut-off value, in the present example of 100 nM. Consequently, no patient with a benignant disease would be within the range of malignant tumour diagnosis, resulting in a 100% specificity.

In the present example, the sensitivity of the test is 93,75%, i.e. those patients being diagnosed as cancer patients actually have a respective malignant disease. This result surpasses all results of previously published tumour marker investigations.

Furthermore, the results show the suitability of the method according to the invention for the distinction between pancreatitis and pancreas carcinoma. The patient group with a carcinoma (n=13, concentration range 83,69-5133 nM) displayed a nearly 250-fold higher median with 1410 versus 5,684 nM TAP in case of a chronic pancreatitis (n=6, concentration range: 0,0080-22,37 nM). Thus, a clear distinction between both diseases was possible.

### LITERATURE

1. L. Gordis, E. B. Gold, 1993. In: V.L.W., Go, E. P. Di Magno, J. D. Gardner, E. Lebenthal, H.A. Reber, G.A. Sheele (Eds.), The Pancreas: Biology, Pathobiology and Disease, 2nd Ed. Raven Press, New York, pp.837-855.

2. WhitcombDC, ApplebaumS, Martin-S P: Hereditary pancreatitis and pan-creatic carcinoma. Ann-N-Y-Acad-Sci. 880 (1999): 201-9

3. Safi F, Beger H G, Bittner R, Büchler M, Krautzberger W. CA 19-9 and pancreatic adenocarcinoma. Cancer 1986;57:779-783.

4. Rötlin M A, Joller H, Largiader F. CA 242 is a new tomour marker for pancreatic cancer. Cancer 1992;71:701-707.

5. Hyöty M, Hyöbty H, Aaran R K, Airo I, Nordback I. Tumor antigens CA 195 and CA 19-9 in pancreatic juice and serum for the diagnosis of pancrea-tic carcinoma. Eur J Surg 1992;158:173-179.

6. Fuzhou T, Appert H E, Myles J, Howard J M: Prognostic value of serum CA 19-9 levels in pancreatic adenocarcinoma. Ann Surg 215(1992): 350-355.

7. Grosfeld J F, Clatworthy H W, Hamoundi AB: Pancreatic malignancy in children. Arch Surg 101 (1970): 370-374

8. Robey G, Danemann A, Martin D J: Pancreatic carcinoma in a neonate. Pediatr Radiol 13 (1983): 284-286

9. Knyrim K In: Claasen/Diehl/Kochsiek Innere Medizin; Urban & Schwarzenberg, Müchen—Wien—Baltimore 1991: 625-627.

10. Brockmann J, Glodny B, Menzel J, Winde G, Senninger N. Quantificating expression of tumor markers in gallblader bile for identification of malignancies of the subhepatic bilio-pancreatic sistem. Langenbecks Archives of Surgery 1999; Suppl I (Forumsvorträge):307-311

11. Terada T, Nakamuna Y. Expression of Pancreatic Enzymes (a-Amylase, Trypsinogen and Lipase) During Human Liver Development and Matu-ration. Gastroenterol 1995;108:1236-1245

12. Ewe K, Karbach U In: Schmidt/Thews (Hrsg) Physiologie des Men-schen; Springer-Verlag Heidelberg-London—Paris- Tokyo; 23. Aufl. 1986

13. Löffler G In: Löffler-Petrides (Hrsg) Physiologische Chemie 4. Auflage; Springer-Verlag Heidelberg-London-Paris-Tokyo; 1988

14. Opie E L; The etiology of acute hemorrhagic pancreatitis. Amer Jour of the Med Sciences 71 (1901): 182-188

15. Hernandez C A, Lerch M M: Sphicter stenosis and gallstone migration through the biliary tract. Lancet 341 (1993): 1371-73.

16. Langmann J In: Medizinische Embryologie—Die normale menschliche Entwicklung und ihre Fehlbildungen. Georg Thieme Verlag Stuttgrad—New York 7. Aufl.: 271-278

1. Use of the trypsin activating peptide for diagnosing malignant bilio-pancreatic diseases.

2. Use of the trypsin activating peptide according to claim 1, characterised in the diagnosis of one of the following carcinomas: pancreas carcinoma, gall-bladder carcinoma, bile-duct cancer, papillary carcinoma.

3. Use of the trypsin activating peptide for diagnosing inflammatory gall-bladder- or bile-duct diseases or inflammations of the papilla Vateri.

4. Use of the trypsin activating peptide for evaluating the benignant or malignant character of bilio-pancreatic diseases.

5. Use of the trypsin activating peptide according to one of the above mentioned claims, characterised in a quantitative detection of the trypsin activating peptide.

6. Use of the trypsin activating peptide according to one of the above mentioned claims, characterised in, that the detection is carried out in blood, urine or bile secretion.

7. Use of the trypsin activating peptide according to claim 6, characterised in an immunological quantification of the trypsin activating peptide.
